(19) United States
(12) Patent Application Publication
(10) Pub. No.: US 2015/0374346 A1
(43) Pub. Date: Dec. 31, 2015

(54) ULTRASOUND DISPLAY CLIENT

(71) Applicant: B-K MEDICAL APS, Herlev (DK)

(72) Inventors: Jesper Lomborg Manigoff, Frederiksberg C (DK); Michael Knud Eibye, Ballerup (DK)

(21) Appl. No.: 14/766,448

(22) PCT Filed: Feb. 15, 2013
(86) PCT No.: PCT/IB2013/000193

§ 371 Date: Aug. 6, 2015

(51) Int. Cl. A61B 8/00; A61B 8/08

(52) U.S. Cl. CPC A61B 8/565; A61B 8/461; A61B 8/469; A61B 8/54; A61B 8/5207

(57) ABSTRACT

An ultrasound imaging system (102) includes an ultrasound scanner (104) and a client device (106) with a display (120). The client device is not a mechanical component of the scanner. The client device requests ultrasound data from the scanner and displays the requested data with the display. A method includes receiving a signal from a client device wherein the client device is not a mechanical component of the scanner, and interpreting a command indicative of the signal, and at least one of: setting a scan parameter of the ultrasound scanner for a scan, transmitting application software to the client device, or transmitting ultrasound data to the client device. A method includes transmitting a signal to an ultrasound scanner, wherein the client device is not a mechanical component of the scanner, and receiving in response to the signal, at least one of: application software or ultrasound data.

### TECHNICAL FIELD

The following generally relates to ultrasound and more particularly to an ultrasound scanner and display client, and is described with particular application to ultrasound imaging; however, the following is also amenable to one or more other ultrasound apparatuses.

### BACKGROUND

Ultrasound (US) imaging has provided useful information about the interior characteristics of an object or subject under examination. An ultrasound imaging scanner has included a transducer array of one or more transducer elements that produces and transmits ultrasound signals and receives ultrasound data produced in response to the ultrasound signals interacting with structure, hardware and/or software that processes the received ultrasound data generating one or more images, and a display that visually presents the one or more images.

With a hand held ultrasound imaging scanner, the transducer array, the hardware and/or software, and the display have been housed in a same housing, which can be carried around by a user in one or both hands and utilized to scan a subject or object. In another configuration, the transducer array is included in a probe that connects, via a cable or the like and/or wirelessly, to a local computing system such as a console or the like, which includes the hardware and/or software that processes the ultrasound data and generates the images. The display may be part of the console or separate from the console and connected thereto via cable, and is used to display the images.

Generally, ultrasound scanners, such as those discussed above, e.g., have dedicated systems that are not be used for any other purposes than ultrasound. Unfortunately, such ultrasound scanners provide little flexibility on standardizing displays throughout hospitals, clinics, etc. Furthermore, such ultrasound scanners do not easily allow multiple views and/or co-viewing via multiple displays in multiple locations. Moreover, dedicated displays are not well-suited to detach from the scanner for optimal ergonomics and/or change in work-position (e.g., from scanning at patient to measurement and review at a desk, or the like).

### SUMMARY

Aspects of the application address the above matters, and others.

In one aspect, an ultrasound imaging system includes an ultrasound scanner and a client device with a display, wherein the client device is not a mechanical component of the scanner, and wherein the client device requests ultrasound data from the scanner and displays the requested data, in response to receiving the requested data, with the display.

In another aspect, a method includes receiving, at an ultrasound scanner, a signal from a client device, wherein the client device is not a mechanical component of the scanner, interpreting, at the ultrasound scanner, a command indicative of the signal, at least one of, in response to the interpreted signal, setting a scan parameter of the ultrasound scanner for a scan, transmitting application software to the client device, or transmitting ultrasound data to the client device.

In another aspect, a method includes transmitting, with a client device, a signal to an ultrasound scanner, wherein the client device is not a mechanical component of the scanner, and receiving, at the client, in response to the signal, at least one of: application software or ultrasound data.

Those skilled in the art will recognize still other aspects of the present application upon reading and understanding the attached description.

### BRIEF DESCRIPTION OF THE DRAWINGS

The application is illustrated by way of example and not limitation in the figures of the accompanying drawings, in which like references indicate similar elements and in which:

FIG. 1 schematically illustrates an example ultrasound imaging system with an ultrasound scanner and one or more client devices with displays;

FIG. 2 illustrates an example of FIG. 1 in which ultrasound scanner includes a console with control and/or data processing services;

FIG. 3 illustrates an example of FIG. 1 in which ultrasound scanner includes a computing cluster with the control and/or data processing services;

FIG. 4 illustrates example method in accordance with the example ultrasound imaging system of FIG. 1.

FIG. 5 illustrates another example method in accordance with the example ultrasound imaging system of FIG. 1.

### DETAILED DESCRIPTION

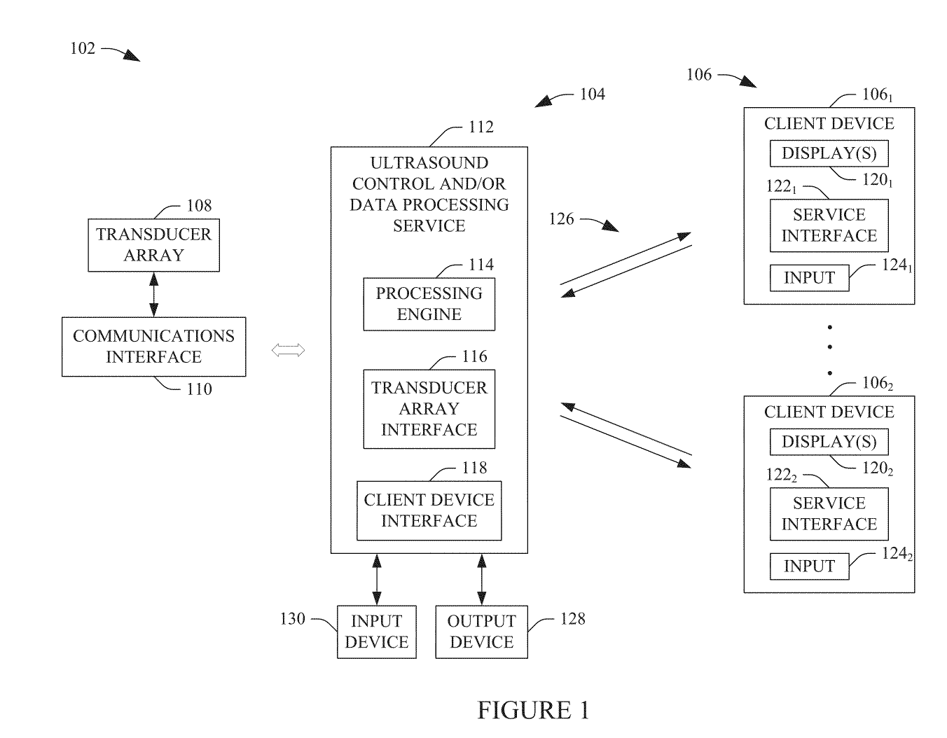

FIG. 1 schematically illustrates an example ultrasound (US) system 102. The ultrasound system 102 includes at least one ultrasound scanner 104 and one or more client devices 106₁, . . . , 106_{N} (collectively referred to herein as client devices 106), where N is an integer equal to or greater than one.

The at least one ultrasound scanner 104 includes a transducer array 108. The transducer array 108 can be a one dimensional (1D) or two dimensional (2D) transducer array with one or more transducer elements. The transducer array 108 is in electrical communication with a communications interface 110.

The at least one ultrasound scanner 104 further includes an ultrasound control and/or data processing service 112. The illustrated ultrasound control and/or data processing service 112 includes an ultrasound processing engine 114 (e.g., with hardware and/or software for processing ultrasound signals received by the transducer array 108 and generating data and/or images based thereon), a transducer array interface 116, and a client device interface 118.

The ultrasound control and/or data processing service 112 can be part of a physical console of the at least one ultrasound scanner 104 and/or part of a park(s) of interconnected servers making up computing clusters, such as a “cloud” based system or like. An output device 128 such as a display monitor and/or an input device 130 such as a keyboard, a keypad, a mouse, a touchscreen, etc. may be part of and/or used with the at least one ultrasound scanner 104.

Where the at least one ultrasound scanner 104 is a hand held scanner, the ultrasound control and/or data processing service 112 and the output and/or input devices 128 and 130 are housed in a same housing. Where the at least one ultrasound scanner 104 is not a hand held scanner, the ultrasound control and/or data processing service 112 and the transducer array 108 are housed in separated devices, with a cable or wireless communications path there between.

In the later instance, the ultrasound control and/or data processing service 112 can be housed in a console. The output and/or input devices 128 and 130 are either integrated with the console or connected thereto with a cable or wireless communications path. The transducer array 108 and communications interface 110 may be housed in a housing such as probe or the like. The communication interface 110 can include a port for connecting a communications cable thereto or a hard-wired communications cable, which are configured to also connect to the interface 116. Additionally or alternatively, the interfaces 110 and 116 communicate over a wireless communications path.

Other ultrasound scanner configurations are also contemplated herein. The one or more client devices 106₁, . . . , 106_{N} respectively include one or more sets of displays 120₁, . . . , 120_{N} (collectively referred to herein as sets of displays 120). In one non-limiting instance, at least one of the displays is part of a computing system, such as a computer, a smartphone, a cellular phone, a tablet computer, an intelligent monitor, etc.). It is to be understood that the one or more client devices 106₁, . . . , 106_{N} are not dedicated physical and/or mechanical components of the scanner 104; the one or more client devices 106₁, . . . , 106_{N} are non-ultrasound devices with displays that can display ultrasound data generated by the scanner 104.

The one or more client devices 106₁, . . . , 106_{N} also respectively include one or more service interfaces 122₁, . . . , 122_{N} (collectively referred to herein as service interfaces 122). In the illustrated embodiment, the service interfaces 122 and the device interface 118 are configured to communicate with each other. The one or more client devices 106₁, . . . , 106_{N} also respectively include one or more input components (inputs) 124₁, . . . , 124_{N} (collectively referred to herein as inputs 124). In the illustrated embodiment, the inputs 124 allow a user to interact with the client device 106.

As described in greater detail below, in one instance, the one or more client devices 106 receive low data rate input (e.g., less than 10 kbits/second and/or other data rate) via the inputs 124 and transmits a signal indicative thereof to the ultrasound control and/or data processing service 112 over low data rate paths 126 through the service and client device interfaces 122 and 118. Alternatively or additionally, the ultrasound control and/or data processing service 112 processes ultrasound signals generated by the transducer array 108, producing processed ultrasound data (e.g., images), and transmits the ultrasound data and/or the processed ultrasound data over the low data rate paths 126, via a low latency stream or the like, to the one or more client devices 106 through the service and client device interfaces 118 and 122.

With such a configuration, the one or more client devices 106 can include consumer grade equipment, standard off the shelf equipment, and/or low cost low power dedicated equipment, etc. which can be used to display ultrasound data in a manner similar or the same as a display of the output device 128 integrated as part of and/or used in connection with the scanner 104. Furthermore, multiple client devices of the one or more client devices 106 can receive and display the same data. For example, one of the one or more client devices 106 may be utilized in the room in which the scan is being performed while another of the one or more client devices 106 is utilized in an image reading room and displays the same processed data. Alternatively, the same raw data can be processed via two different processing chains of the processing service 112 and displayed on different client devices 106.

The one or more client devices 106 display the data via application software executing on the one or more client devices 106 such as a web client, a web browser, an applet, or the like. In one instance, the application software invokes instantiation of a graphical user interface (GUI) with one or more view ports for visually presenting ultrasound data. Optionally, the GUI may include drop down menus, control buttons, and/or other activateable soft controls. The application software can be stored on the one or more client devices 106 and/or downloadable, e.g., via a network from the service 112, portable memory, and/or other source. This allows for personalized user interfaces as the application software could be loaded on demand and/or on the fly based on the particular device 106 and/or operator.

In one instance, the client devices 106 and the ultrasound control and/or data processing service 112 are entities on a network. In this instance, a user of a client device 106 can locate a transducer 108 of interest, a service 112 of interest, and/or data of interest, e.g., by patient identification, clinician identification, transducer probe identification, examination room identification, etc., via the network and request the data from the ultrasound control and/or data processing service 112. Alternatively, a user can located a client device 106 of interest, e.g., by client device identification, identification of an operator of the client device, etc., via the network and request transmission of data to the client device 106. In either instance, this may require a user name and password and/or other authorization information.

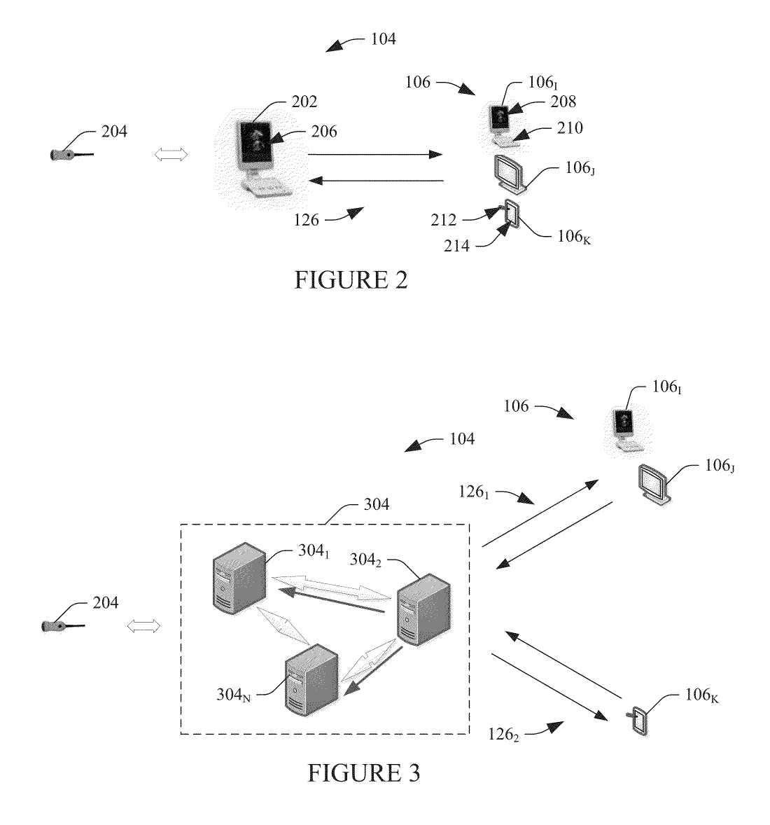

FIG. 2 illustrates an example in which the scanner 104 includes a console 202 with at least a sub-portion of the ultrasound control and/or data processing engine 112 (FIG. 1) and a probe 204.

In the illustrated example, the console 202 may include transmit and receive circuitry. The transmit circuitry controls excitation of the transducer elements of the transducer array, which allows for steering and/or focusing the transmitted beam from predetermined origins along the array and at predetermined angles, and the receive circuitry receives ultrasound data produced in response to beam interacting with structure generates and a signal indicative thereof.

The receive circuitry (and/or other circuitry) may include circuitry that delays and sums received ultrasound data. Other suitable processing includes, but is not limited to, spatial compounding, filtering (e.g., FIR, IIR, etc.), color flow, Doppler, synthetic aperture, 2D, 3D, 4D, and/or other processing. The console 202 may also include an image processor that convert the processed data to generate data for display, for example, by converting the data to the coordinate system of an integrated display 206 and/or other display. In another embodiment, the display 206 is omitted.

In this example, the probe 204 includes the transducer array 108 (FIG. 1) and the communications interface 110 (FIG. 1). Furthermore, multiple of the one or more client devices 106, namely client devices 106_{I}, 106_{J}, and 106_{K}, are used in connection with the scanner 104. In this embodiment, the multiple of the one or more devices 106_{I}, 106_{J}, and 106_{K}, are different devices, with the 106_{K} being a console similar to the console 106 with a keyboard 210 and a display 208, the 106_{J} being a touchscreen display, and the 106_{K} being a smartphone with a stylus 212 and stylus activate display 214.

The one or more client devices 106 transmits signals over the communication path 126, via the service interface 122 (FIG. 1) and the client device interface 118 (FIG. 1), to the console 202. The signal, in one example, is indicative of a user input, such as a touch of a touch sensitive region of a touch screen display, press of a button of a keyboard or keypad, click of a mouse over a particular region of the display, etc., which invokes transmission of the control signal. For example, pressing the key labeled “A” may invoke a signal unique to that key. In this instance, the key labeled “A” corresponds to a predetermined act by the service 112, but the signal only includes the signal unique to key labeled “A” and not the act.

The console 202 transmits application software and data (raw and/or processed) over the communication path 126, via the device interface 118 (FIG. 1) and the service interface 122 (FIG. 1), to the multiple client devices 106. The console 202, in response to receiving the signal transmitted by the one or more client devices 106, transmits the application software and/or the data, is set up to perform a scan (e.g., setting at least one scan parameter), excites the transducer array 108 to transmit and/or receive ultrasound signals for a scan, etc. A look up table or other approach can be used to interpret the signal by mapping the signal to a command via a predetermined mapping.

As discussed herein, the application software may invoke a GUI with one or more view ports for visually presenting ultrasound data. Such a GUI may include drop down menus, control buttons, and/or other features. Furthermore, the application software may include a web browser, an applet, or the like. The application software can be stored and ran on the one or more client devices 106. Alternatively, the application software can be executed over a network from the console 202 and/or other component. In either instance, the application software can be loaded on demand and/or on the fly based on the particular client device 106 and/or operator.

Where the signal includes low data rate input like data representing the pressed key, the communication path 126 may be a low data rate communications path. By way of example, the one or more client devices 106 can package the signal and transmit the packaged signal over a TCP/IP based or other network. The console 202 can package the data into a low latency stream (e.g., compressed video streaming feeds) that can be decoded and/or decompressed by decoding and/or decompressing components of the one or more client devices 106, without need for traditional ultrasound processing by the client devices 106.

FIG. 3 illustrates an example in which the scanner 104 includes a cluster of computing systems 304₁, 304₂, . . . , 304_{N} (collectively referred to herein as cluster of computing systems 304) that implement at least a sub-portion of the ultrasound control and/or data processing engine 112 (FIG. 1) and a probe 204.

In this example, the probe 204 and/or the cluster of computing systems 304 includes the transmit circuitry and/or the receive circuitry, and the cluster of computing systems 304 processes received ultrasound data. Such processing may include the processing discussed in connection with FIG. 2 and/or other processing. The probe 204 may include an integrated display.

One or more of the computing systems 304₁, 304₂, . . . , 304_{N} can be used individually and/or in combination to process received ultrasound data, including processing the data via two or more different processing chains, each utilizing a different set of processing algorithms and/or resources and/or computing systems 304₁, 304₂, . . . , 304_{N}. In the illustrated example, data processed via a first processing channel is transmitted to client devices 106_{I} and 106_{J}, and data processed via a second processing channel is transmitted to client device 106_{K}.

The one or more client devices 106 transmit signals over the communication paths 126₁ and 126₂, via the service interface 122 (FIG. 1) and the device interface 118 (FIG. 1), to the cluster of computing systems 304. Similar to FIG. 2, the signal, in one embodiment, is indicative of a user input, such as a touch of a touch sensitive region of a touch screen display, press of a button of a keyboard or keypad, click of a mouse over a particular region of the display, etc., which invokes transmission of the control signal. For example, clicking with mouse, pressing with a stylus or finger, etc. on a touch screen region may invoke a signal unique to that location.

The cluster of computing systems 304, like the console 202 (FIG. 2), transmits application software and data (raw and/or processed) over the communication paths 126₁ and 126₂, via the device interface 118 (FIG. 1) and the service interface 122 (FIG. 1), to the multiple of the one or more client devices 106. The cluster of computing systems 304, in response to receiving the signal transmitted by the one or more client devices 106, transmits the application software and/or the data, is set up to perform a scan, excites the transducer array 108 (FIG. 1) to transmit and/or receive signals for a scan, etc.

Where the signal includes low data rate input like data representing the touchscreen location, the communication paths 126₁ and 126₂ may include a low data rate communication path. The cluster set of computing systems 304 can package the data into a low latency stream that can be decoded and/or decompressed by decoding and/or decompressing components of the one or more client devices 106. Likewise, the probe 204 can package the data into a low latency stream that can be decoded and/or decompressed by the cluster of computing systems 304.

With respect to the client devices 106, the application software may include a GUI with one or more view ports for visually presenting ultrasound data and drop down menus, control buttons, and/or other features. Furthermore, the application software may include a web browser, an applet, or the like. The application software can be stored and ran on the one or more client devices 106. Alternatively, the application software can be executed over a network. In either instance, the application software can be loaded on demand and/or on the fly based on the particular client device 106 and/or operator.

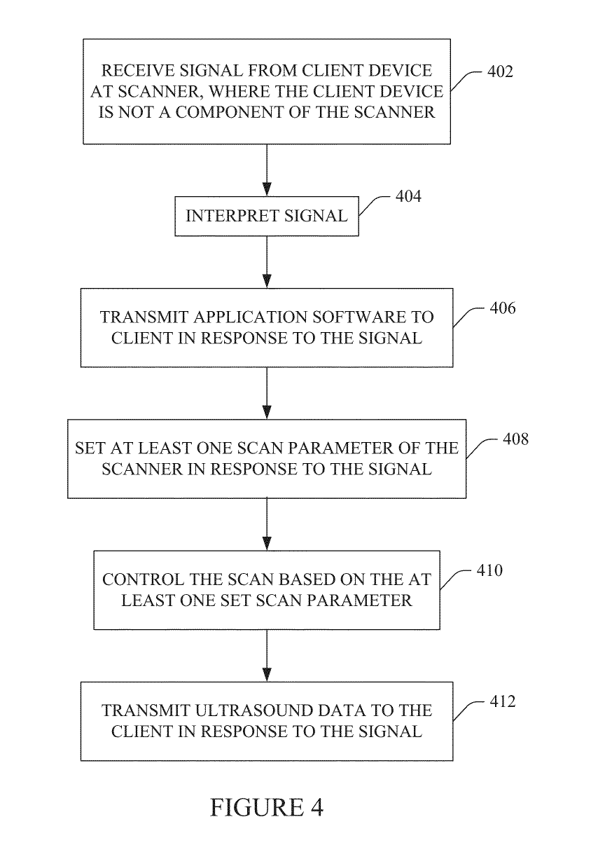

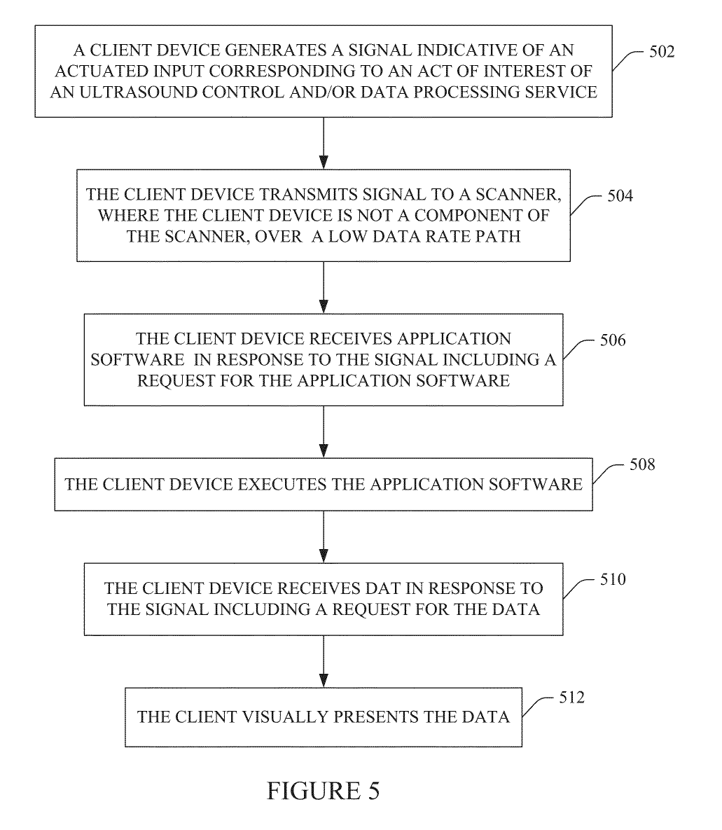

FIGS. 4 and 5 illustrate example methods in accordance with the description herein.

It is to be appreciated, for any or all of the methods, that the order of the acts is provided for explanatory purposes and is not limiting. As such, one or more of the acts may occur in a different order. Furthermore, one or more of the acts may be omitted and/or one or more additional acts may be added.

FIG. 4 illustrate an example method in which a client device 106 communicates with the ultrasound control and/or data processing device 112.

At 402, a signal from a client device 106 is received at the ultrasound control and/or data processing device 112. As described herein, the ultrasound control and/or data processing device 112 may be part of a physical ultrasound scanner and/or a cluster of computing devices.

At 404, the ultrasound control and/or data processing device 112 interprets the signal. As discussed herein, in one instance, this includes mapping the signal to a command in a look up table or the like. As described herein, the signal may include information to set up the ultrasound control and/or data processing device 112 for a scan (e.g., a scan parameter) and/or a request to receive one or more of application software or data (raw and/or processed). For explanatory purposes, this example will cover both scenarios.

At 406, where the command includes a request for application software, the ultrasound control and/or data processing device 112 transmits application software to the client device 106. As discussed herein, the application software can be specific to the transducer, the clinician, and/or other information.

At 408, where the command includes set up information for a scan, the ultrasound control and/or data processing device 112 utilizes this information to set up a scan. Where act 406 is included, this information may alternatively be received via the application software.

At 410, the ultrasound control and/or data processing device 112 controls the scan based on the set up information.

At 412, where the command includes a request for data, the ultrasound control and/or data processing device 112 transmits raw and/or processed data to the client device 106. Where act 406 is included, the request for this information may alternatively be received through the application software.

Turning to FIG. 5, an example method is illustrated in which the ultrasound control and/or data processing device 112 communicates with a client device 106.

At 502, a client device 106 receives in input, via an input device 124, and generates a signal indicative thereof. As discussed herein, the input may correspond to a particular act of interest of an ultrasound scanner 104, but only indicates the key, touchscreen, etc. actuated at the client device 106.

At 504, the client device 106 transmits the signal to the ultrasound scanner 104. As discussed herein, the client device 106 is not a mechanical component of the scanner. In addition, the signal includes at least one of a scan setup parameter or a request for at least one of application software or ultrasound data and can be conveyed over a low data rate communications path.

At 506, in response to the signal including a request for the application software, the client device 106 receives the application software, which is transmitted by the ultrasound control and/or data processing system 112.

At 508, the client device 106 executes the application software.

At 510, in response to the signal including a request for the data, the client device 106 receives the data, which is transmitted by the scanner 104.

At 512, the client device displays the data via a display of the client device.

The above may be implemented in connection with computer readable instructions, encoded or embedded on computer readable storage medium, which, when executed by a computer processor(s), cause the processor(s) to carry out one or more of the described acts. Additionally or alternatively, at least one of the computer readable instructions is carried by a signal, carrier wave or other transitory medium.

The application has been described with reference to various embodiments. Modifications and alterations will occur to others upon reading the application. It is intended that the invention be construed as including all such modifications and alterations, including insofar as they come within the scope of the appended claims and the equivalents thereof.

1. An ultrasound imaging system, comprising:
an ultrasound scanner; and
a client device with a display, wherein the client device is not a component of the scanner, and wherein the client device requests ultrasound data from the scanner and displays the requested data, in response to receiving the requested data, with the display.

2. The system of claim 1, wherein the client device requests application software from the scanner.

3. The system of claim 2, wherein the request is transmitted over a low data rate path to the scanner.

4. The system of claim 1, wherein the scanner conveys the application software to the client device.

5. The system of claim 2, wherein the application software corresponds to a particular transducer of the scanner or a particular clinician.

6. The system of claim 2, wherein the client device executes the application software.

7. The system of claim 6, wherein the client device request the data through the executing application software.

8. The system of claim 6, wherein the client device displays the data via the executing application software.

9. The system of claim 1, wherein the scanner packages at least one of the application software or the data in at least one of an encoded or compressed video feed.

10. The system of claim 9, wherein the at least one of the encoded or the compressed video stream feed is transmitted over a low latency path.

11. The system of claim 9, wherein the client device at least one of decodes or decompresses the video feed.

12. The system of claim 1, further comprising:
at least a second client device with a second display, wherein the second client device is not a physical component of the scanner, and wherein the client device and the second client device display the same data.

13. The system of claim 1, further comprising:
at least a second client device with a second display, wherein the second client device is not a physical component of the scanner, and wherein the scanner processes the data with different processing chains and transmits first processed data to the client device and second different processed data to the second client device.

14. The system of claim 1, wherein the client device is one of a computer, a smartphone, a cellular phone, a tablet computer, or an intelligent monitor, etc.

15. A method, comprising:
receiving, at an ultrasound scanner, a signal from a client device, wherein the client device is not a mechanical component of the scanner;
interpreting, at the ultrasound scanner, a command indicative of the signal; and
at least one of, in response to the interpreted signal, setting a scan parameter of the ultrasound scanner for a scan, transmitting application software to the client device, or transmitting ultrasound data to the client device.

16. The method of claim 15, wherein interpreting the signal includes mapping the signal to a set of predetermined commands in a look up table.

17. The method of claim 15, wherein the signal includes a value indicative of a pressed key, an actuated region of a touchscreen, or a region of a screen selected with a computer pointer.

18. The method of claim 15, wherein the application software corresponds to a particular transducer of the scanner or a particular clinician.

19. The system of claim 15, where the data includes processed raw data, and further comprising:
processing the raw data with a single processing chain; and
transmitting the processed data to at least two client devices.

20. The method of claim 15, where the data includes processed raw data, and further comprising:
processing the raw data with at least two processing chains; and
transmitting the processed data to at least two client devices.

21. The method of claim 15, further comprising:
receiving the signal over a low data rate path; and
transmitting the application software or the ultrasound data as a low latency video stream.

22. A method, comprising:
transmitting, with a client device, a signal to an ultrasound scanner (104), wherein the client device is not a mechanical component of the scanner;
receiving, at the client, in response to the signal, at least one of: application software or ultrasound data.

23. The method system of claim 22, further comprising:
executing the application software; and
transmitting, to the scanner, a request for the ultrasound data.

24. The method system of claim 23, further comprising:
receiving, at the client device, the requested ultrasound data; and
visually presenting, at the client device, the received ultrasound data.

25. The method system of claim 24, wherein the ultrasound data is raw unprocessed data

26. The method system of claim 24, wherein the same ultrasound data is processed data.

27. The method system of claim 23, wherein the same ultrasound data is also transmitted to another client device.

28. The method system of claim 23, wherein the ultrasound data includes a same set of raw data processed via two different processing chains, and where the differently processed data transmitted to two different client devices.

29. The method of claim 22, further comprising:
transmitting the signal over a low data rate path; and
receiving the at least one of: application software or ultrasound data as a low latency video stream.
